(19) United States
(12) Patent Application Publication
(10) Pub. No.: US 2017/0281026 A1
(43) Pub. Date: Oct. 5, 2017

(54) BIOMETRIC SENSOR FOR DETERMINING HEART RATE USING PHOTOPLETHYSMOGRAPH

(71) Applicant: DAQRI, LLC, Los Angeles, CA (US)

(72) Inventors: Teresa Ann Nick, Woodland Hills, CA (US); Laura Berman, Venice, CA (US); Arye Barnehama, Venice, CA (US)

(21) Appl. No.: 15/089,293

(22) Filed: Apr. 1, 2016

(51) Int. Cl. A61B 5/024; G02B 27/01; A61B 5/00; G06T 11/00

(52) U.S. Cl. CPC A61B 5/02416; G06T 11/00; G02B 27/017; A61B 5/7225; A61B 5/742; A61B 5/6803; G02B 2027/0178

(57) ABSTRACT

A wearable computing device includes a display device for displaying augmented reality (AR) images and a biometric sensor for obtaining a heartrate of a user wearing the wearable computing device. The biometric sensor includes a photosensor that emits a light into the surface of a body part of the user. Using photoplethysmography, the photosensor measures voltages from the light reflected from or transmitted through the user's body part at a sampling rate of at least 100 Hz. The measured voltages are then filtered and normalized. Slopes for the resulting set of voltages are then determined on a sliding window basis of approximately 90 milliseconds (ms). Interpulse intervals are then determined for consecutive local minima within each set of sliding windows. The biometric sensor then computes a real-time heartrate for the user from the interpulse intervals, which may then be displayed on the display device of the wearable computing device.

### TECHNICAL FIELD

The subject matter disclosed herein generally relates to a biometric monitor and, in particular, to a biometric sensor configured to determine heart rate using photoplethysmography and the interpretation of one or more determined voltage events into corresponding interpulse intervals.

### BACKGROUND

Augmented reality (AR) is a live direct or indirect view of a physical, real-world environment whose elements are augmented (or supplemented) by computer-generated sensory input such as sound, video, graphics or Global Positioning System (GPS) data. With the help of advanced AR technology (e.g., adding computer vision and object recognition) the information about the surrounding real world of the user becomes interactive. Device-generated (e.g., artificial) information about the environment and its objects can be overlaid on the real world.

Typically, a user uses a computing device to view the augmented reality. The computing device may be a wearable computing device used in an environment where the user's health is an important consideration. The computing device may also include a biometric sensor that monitors information about the user's health, such as the user's heartrate. However, conventional biometric sensors use a non-trivial amount of computing resources (e.g., electric power and memory) and physical resources (e.g., physical space within the computing device) to monitor this information. Where computing and physical resources are factors in designing a wearable computing device that provides an augmented reality view of an environment, implementing a biometric sensor that efficiently uses such resources can be a technically challenging task.

### BRIEF DESCRIPTION OF THE DRAWINGS

Some embodiments are illustrated by way of example and not limited to the figures of the accompanying drawings.

FIG. 1 is a block diagram illustrating an example of a network environment suitable for a wearable computing device, according to an example embodiment.

FIG. 2 is a block diagram of a biometric sensor, according to an example embodiment.

FIG. 3 illustrates a graph of measured voltages obtained by a photosensor of the biometric sensor illustrated in FIG. 2, according to an example embodiment.

FIG. 4 illustrates a graph of determined slopes, according to an example embodiment, corresponding to the measured voltages illustrated in the graph of FIG. 3.

FIG. 5 illustrates a graph of interpulse intervals, according to an example embodiment, corresponding to the measured voltages illustrated in the graph and derived from the times of the peak slopes illustrated in the graph of FIG. 4.

FIGS. 6A-6B illustrate a method for determining a heartrate using the biometric sensor illustrated in FIG. 2, according to an example embodiment.

FIG. 7 is a block diagram illustrating components of a machine, according to some example embodiments, able to read instructions from a machine-readable medium (e.g., a machine-readable storage medium) and perform any one or more of the methodologies discussed herein.

### DETAILED DESCRIPTION

The disclosure provides for a biometric sensor that determines one or more heartrates using photoplethysmography and the interpretation of one or more determined voltages into corresponding interpulse intervals. In one embodiment, the biometric sensor includes machine-readable memory storing computer-executable instructions, and at least one hardware processor in communication with the machine-readable memory that, when the computer-executable instructions are executed, configures the biometric sensor to obtain a plurality of voltages in response to a photosensor emitting light into a surface of a human body, and filter at least one predetermined frequency from the plurality of voltages to obtain a plurality of filtered voltages. The biometric sensor is further configured to normalize the plurality of filtered voltages to obtain a plurality of normalized voltages, determine a plurality of slopes based on the plurality of normalized voltages, and determine a plurality of local minima based on the determined plurality of slopes. In addition, the biometric sensor is configured to determine a plurality of interpulse intervals based on the plurality of local minima, wherein at least one interpulse interval represents a time between a first local minima selected from the plurality of local maxima and a consecutive, second local minima selected from the plurality of local minima, determine at least one heartrate from the determined plurality of interpulse intervals, and communicate the determined at least one heartrate to a display.

In another embodiment of the biometric sensor, the at least one predetermined frequency is filtered from the plurality of voltages using at least one bandpass infinite impulse response filter.

In a further embodiment of the biometric sensor, the at least one predetermined frequency comprises a range of frequencies from approximately 1 Hz to approximately 50 Hz

In yet another embodiment of the biometric sensor, the biometric sensor is further configured to determine a median voltage from the plurality of filtered voltages, and adjust each voltage of the plurality of filtered voltages by the determined median voltage.

In yet a further embodiment of the biometric sensor, the plurality of slopes occur within a preconfigured time duration, and the preconfigured time duration is changed by a predetermined amount in response to a determination that the number of the plurality of slope minima occurring within the preconfigured time duration is less than a minimum threshold limit or greater than a maximum threshold limit.

In another embodiment of the biometric sensor, the plurality of filtered voltages are decimated by a preconfigured amount.

In a further embodiment of the biometric sensor, the plurality of voltages are obtained from the photosensor at a sampling rate of at least 100 Hz.

This disclosure also describes a method for measuring a heart rate through photoplethysmography where the method includes obtaining, by a photosensor, a plurality of voltages in response to emitting light into a surface of a human body, and filtering, by at least one hardware processor, at least one predetermined frequency from the plurality of voltages to obtain a plurality of filtered voltages. The method also includes normalizing, by at least one hardware processor, the plurality of filtered voltages to obtain a plurality of normalized voltages, and determining, by at least one hardware processor, a plurality of slopes based on the plurality of normalized voltages. Furthermore, the method includes determining, by at least one hardware processor, a plurality of local minima based on the determined plurality of slopes, and determining, by at least one hardware processor, a plurality of interpulse intervals based on the plurality of local minima, wherein at least one interpulse interval represents a time between a first local minima selected from the plurality of local minima and a consecutive, second local minima selected from the plurality of local minima. Additionally, the method includes determining, by at least one hardware processor, at least one heartrate from the determined plurality of interpulse intervals, and communicating, using at least one communication interface, the determined at least one heartrate to a display.

In another embodiment of the method, the at least one predetermined frequency is filtered from the plurality of voltages by at least one bandpass infinite impulse response filter.

In a further embodiment of the method, the at least one predetermined frequency comprises a range of frequencies from approximately 1 Hz to approximately 50 Hz.

In yet another embodiment of the method, the method includes determining a median voltage from the plurality of filtered voltages, and adjusting each voltage of the plurality of filtered voltages by the determined median voltage.

In yet a further embodiment of the method, the plurality of slopes occur within a preconfigured time duration, and the preconfigured time duration is changed by a predetermined amount in response to a determination that the number of the plurality of slopes occurring within the preconfigured time duration is less than a minimum threshold amount or greater than a maximum threshold amount.

In another embodiment of the method, the plurality of filtered voltages are decimated by a preconfigured amount.

In a further embodiment of the method, the plurality of voltages are obtained from the photosensor at a sampling rate of at least 100 Hz.

This disclosure also provides for a machine-readable medium having computer-executable instructions stored thereon that, when executed by at least one hardware processor, causes a biometric sensor to perform a plurality of operations, where the plurality of operations include obtaining a plurality of voltages in response to emitting light into a surface of a human body, and filtering at least one predetermined frequency from the plurality of voltages to obtain a plurality of filtered voltages. The plurality of operations also include normalizing the plurality of filtered voltages to obtain a plurality of normalized voltages, and determining a plurality of slopes based on the plurality of normalized voltages. In addition, the operations include determining a plurality of local minima based on the determined plurality of slopes, and determining a plurality of interpulse intervals based on the plurality of local minima, wherein at least one interpulse interval represents a time between a first local minima selected from the plurality of local minima and a consecutive, second local minima selected from the plurality of local minima. Furthermore, the operations include determining at least one heartrate from the determined plurality of interpulse intervals, and communicating the determined at least one heartrate to a display.

In another embodiment of the machine-readable medium, the at least one predetermined frequency is filtered from the plurality of voltages by at least one bandpass infinite impulse response filter.

In a further embodiment of the machine-readable medium, the at least one predetermined frequency comprises a range of frequencies from approximate 1 Hz to approximate 50 Hz.

In yet another embodiment of the machine-readable medium, the plurality of operations further include determining a median voltage from the plurality of filtered voltages, and adjusting each voltage of the plurality of filtered voltages by the determined median voltage.

In yet a further embodiment of the machine-readable medium, the plurality of slopes occur within a preconfigured time duration, and the preconfigured time duration is changed by a predetermined amount in response to a determination that the number of the plurality of slopes occurring within the preconfigured time duration is less than a minimum threshold amount or greater than a maximum threshold amount.

In another embodiment of the machine-readable medium, the plurality of voltages are obtained from the photosensor at a sampling rate of at least 100 Hz.

Unless explicitly stated otherwise, components and functions are optional and may be combined or subdivided, and operations may vary in sequence or be combined or subdivided. In the following description, for purposes of explanation, numerous specific details are set forth to provide a thorough understanding of example embodiments. It will be evident to one skilled in the art, however, that the present subject matter may be practiced without these specific details.

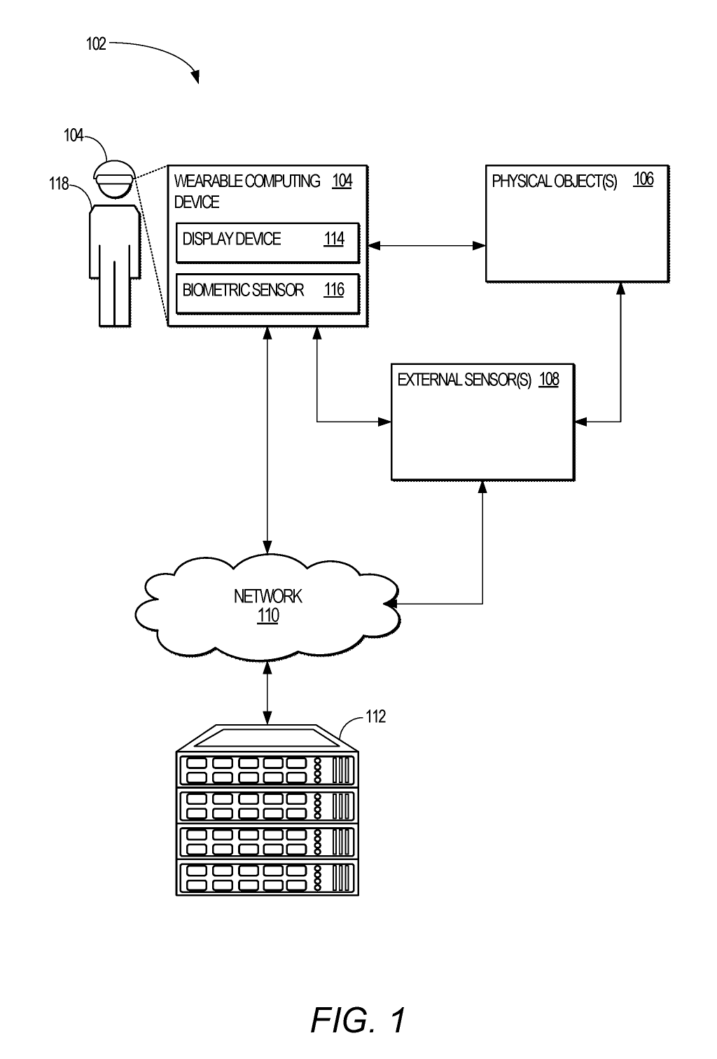

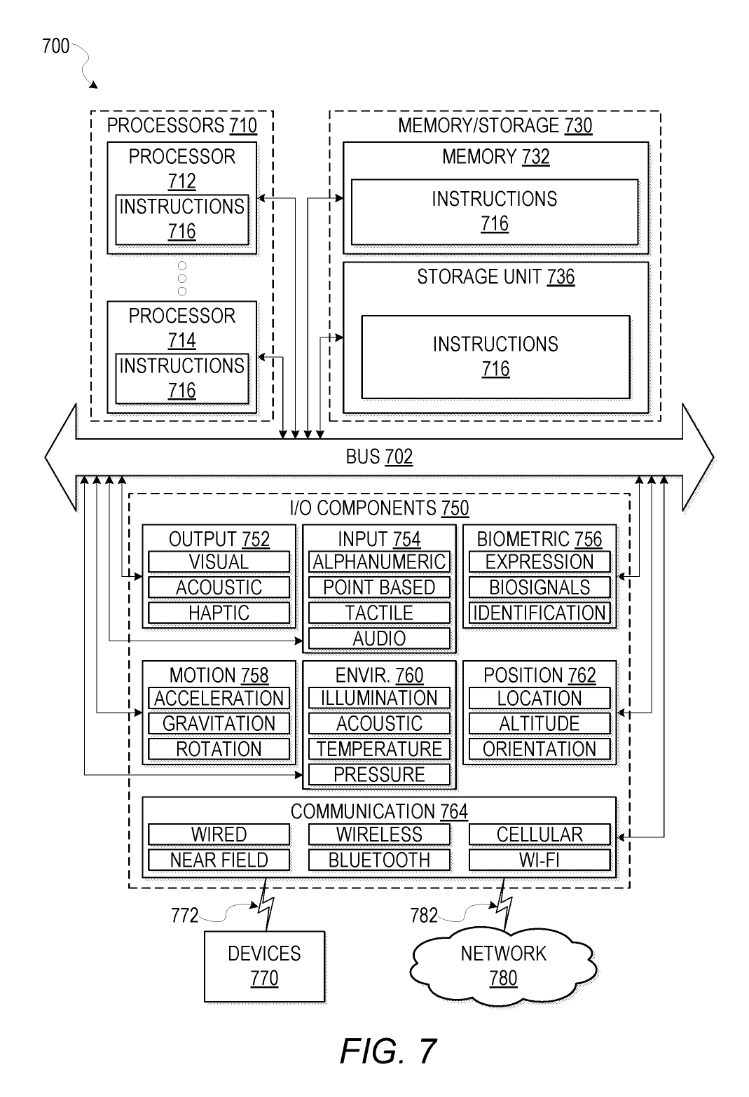

FIG. 1 is a block diagram illustrating an example of a network environment 102 suitable for a wearable computing device 104, according to an example embodiment. The network environment 102 includes the wearable computing device 104 and a server 112 communicatively coupled to each other via a network 110. The wearable computing device 104 further includes a display device 114 and a biometric sensor 116. The wearable computing device 104 and the server 112 may each be implemented in a computer system, in whole or in part, as described below with respect to FIG. 7.

The server 112 may be part of a network-based system. For example, the network-based system may be or include a cloud-based server system that provides additional information, such as three-dimensional (3D) models or other virtual objects, to the wearable computing device 104.

The wearable computing device 104 may be implemented in various form factors. In one embodiment, the wearable computing device 104 is implemented as a helmet, which the user 118 wears on his or her head, and views objects (e.g., physical object(s) 106) through a display device 114, such as one or more lenses, affixed to the wearable computing device. In another embodiment, the wearable computing device 104 is implemented as a lens frame, where the display device 114 are implemented as one or more lenses affixed thereto. In yet another embodiment, the wearable computing device 104 is implemented as a watch (e.g., a housing mounted or affixed to a wrist band), and the display device 114 is implemented as a display (e.g., liquid crystal display or light emitting diode display) affixed to the wearable computing device 104.

A user 118 may wear the wearable computing device 104 and view one or more physical object(s) 106 in a real world physical environment. The user 118 may be a human user (e.g., a human being), a machine user (e.g., a computer configured by a software program to interact with the wearable computing device 104), or any suitable combination thereof (e.g., a human assisted by a machine or a machine supervised by a human). The user 118 is not part of the network environment 102, but is associated with the wearable computing device 104. For example, the wearable computing device 104 may be a computing device with a camera and a transparent display. In another example embodiment, the wearable computing device 104 may be hand-held or may be removably mounted to the head of the user 118. In one example, the display device 114 may include a screen that displays what is captured with a camera (not shown) of the wearable computing device 104. In another example, the display of the display device 114 may be transparent or semi-transparent such as in lenses of wearable computing glasses or the visor or a face shield of a helmet.

The user 118 may be a user of an augmented reality (AR) application executable by the wearable computing device 104 and/or the server 112. The AR application may provide the user 118 with an AR experience triggered by one or more identified objects (e.g., physical object(s) 106) in the physical environment. For example, the physical object(s) 106 may include identifiable objects such as a two-dimensional (2D) physical object (e.g., a picture), a 3D physical object (e.g., a factory machine), a location (e.g., at the bottom floor of a factory), or any references (e.g., perceived corners of walls or furniture) in the real-world physical environment. The AR application may include computer vision recognition to determine various features within the physical environment such as corners, objects, lines, letters, and other such features or combination of features.

In one embodiment, the objects in an image captured by the wearable computing device 104 are tracked and locally recognized using a local context recognition dataset or any other previously stored dataset of the AR application. The local context recognition dataset may include a library of virtual objects associated with real-world physical objects or references. In one embodiment, the wearable computing device 104 identifies feature points in an image of the physical object 106. The wearable computing device 104 may also identify tracking data related to the physical object 106 (e.g., GPS location of the wearable computing device 104, orientation, or distance to the physical object(s) 106). If the captured image is not recognized locally by the wearable computing device 104, the wearable computing device 104 can download additional information (e.g., 3D model or other augmented data) corresponding to the captured image, from a database of the server 112 over the network 110.

In another example embodiment, the physical object(s) 106 in the image is tracked and recognized remotely by the server 112 using a remote context recognition dataset or any other previously stored dataset of an AR application in the server 112. The remote context recognition dataset may include a library of virtual objects or augmented information associated with real-world physical objects or references.

In one embodiment, the wearable computing device 104 also includes a biometric sensor 116 affixed thereto. For example, where the wearable computing device 104 is implemented as a head-mounted device, the biometric sensor 116 may be disposed within an interior surface of the wearable computing device 104 such that the biometric sensor 116 is in contact with the skin of the user's 104 head (e.g., the forehead). As another example, where the wearable computing device 104 is implemented as a wrist-mounted device (e.g., a watch), the biometric sensor 116 may be disposed within, or in contact with, an exterior surface of the wearable computing device 104 such that the biometric sensor 116 is also in contact with the skin of one of the user's 104 limbs (e.g., a wrist of a forearm). In either examples, the biometric sensor 116 is arranged or disposed within the wearable computing device 104 such that it makes contact with the user 104.

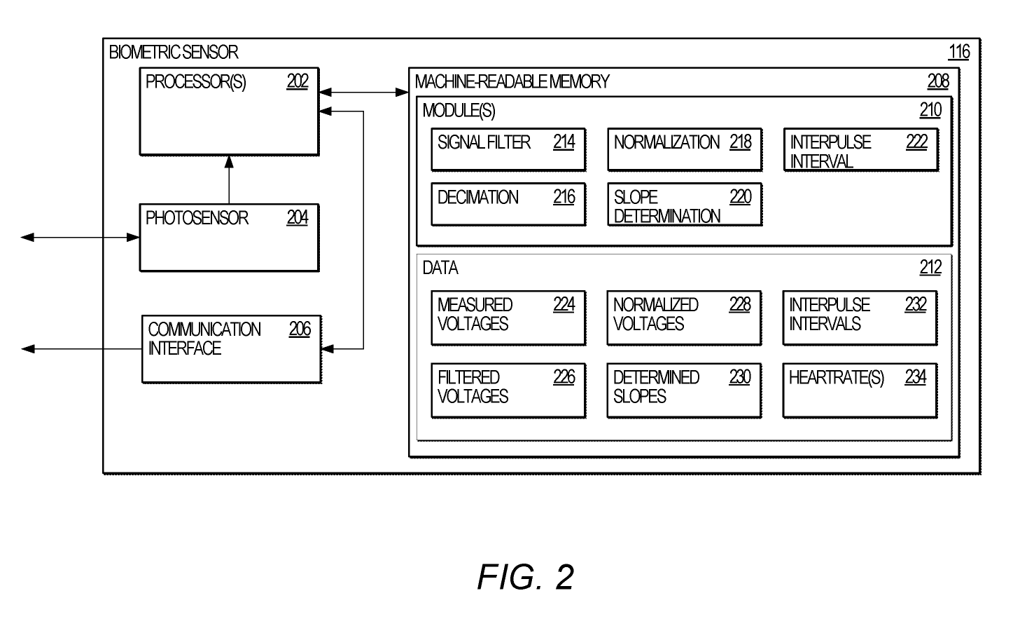

As discussed below with reference to FIG. 2, the biometric sensor 116 is configured to provide a heart rate of the user 104 relatively instantaneously using photoplethysmography and detecting (or identifying) when a heart ventricle contracts. The biometric sensor 116 disclosed herein leverages a light-weight processing technique that determines the user's 104 heart rate within a short time frame (e.g., within seconds). In addition, the disclosed biometric sensor 116 provides a number of benefits in the medical field, such as chronic stress monitoring, irregular heart beat detection, arteriosclerosis measurements, and other such medical concerns. Accordingly, the biometric sensor 116 provides improvements and advancements in other scientific and medical fields, such as cardiology and arteriology.

In one embodiment, the biometric sensor 116 communicates with the display device 114 to display one or more measurements on the display device 114. For example, where the display device 114 is an LED display, the display device 114 may display a resting heart rate obtained from the biometric sensor 116. Further still, where the display device 114 is a lens or other transparent display through which the user 118 views one or more physical object(s) 106, the measurements obtained from the biometric sensor 116 may also be displayed on a lens of the display device 114. Similarly, one or more alerts and notifications generated by the biometric sensor 116 may also be displayed on the display device 114, such as where an irregular heart beat is detected or determined, or where a detected heart beat exceeds (or falls below) a preconfigured heart beat threshold. In these instances, the wearable computing device 104 may be further configured to communicate an alert (e.g., via wireless communication) to a provider of emergency services. Additionally and/or alternatively, the biometric sensor 116 may be configured to communicate wirelessly with one or more devices other than the wearable computing device 104. For example, the biometric sensor 116 may be configured with one or more Uniform Resource Locations (URLs) or Internet Protocol (IP) addresses of other devices that the biometric sensor 116 is to communicate with.

The network environment 102 also includes one or more external sensors 108 that interact with the wearable computing device 104 and/or the server 112. The external sensors 108 may be associated with, coupled to, or related to the physical object(s) 106 to measure a location, status, and characteristics of the physical object(s) 106. Examples of measured readings may include but are not limited to weight, pressure, temperature, velocity, direction, position, intrinsic and extrinsic properties, acceleration, and dimensions. For example, external sensors 108 may be disposed throughout a factory floor to measure movement, pressure, orientation, and temperature. The external sensor(s) 108 can also be used to measure a location, status, and characteristics of the wearable computing device 104 and the user 118. The server 112 can compute readings from data generated by the external sensor(s) 108. The server 112 can generate virtual indicators such as vectors or colors based on data from external sensor(s) 108. Virtual indicators are then overlaid on top of a live image or a view of the physical object(s) 106 (e.g., displayed on the display device 114) in a line of sight of the user 118 to show data related to the physical object(s) 106. For example, the virtual indicators may include arrows with shapes and colors that change based on real-time data. Additionally and/or alternatively, the virtual indicators are rendered at the server 112 and streamed to the wearable computing device 104.

The external sensor(s) 108 may include one or more sensors used to track various characteristics of the wearable computing device 104 including, but not limited to, the location, movement, and orientation of the wearable computing device 104 externally without having to rely on sensors internal to the wearable computing device 104. The external sensor(s) 108 may include optical sensors (e.g., a depth-enabled 3D camera), wireless sensors (e.g., Bluetooth, Wi-Fi), Global Positioning System (GPS) sensors, and audio sensors to determine the location of the user 118 wearing the wearable computing device 104, distance of the user 118 to the external sensor(s) 108 (e.g., sensors placed in corners of a venue or a room), the orientation of the wearable computing device 104 to track what the user 118 is looking at (e.g., direction at which a designated portion of the wearable computing device 104 is pointed, e.g., the front portion of the wearable computing device 104 is pointed towards a player on a tennis court).

Furthermore, data from the external sensor(s) 108 and internal sensors (not shown) in the wearable computing device 104 may be used for analytics data processing at the server 112 (or another server) for analysis on usage and how the user 118 is interacting with the physical object(s) 106 in the physical environment. Live data from other servers may also be used in the analytics data processing. For example, the analytics data may track at what locations (e.g., points or features) on the physical object(s) 106 or virtual object(s) (not shown) the user 118 has looked, how long the user 118 has looked at each location on the physical object(s) 106 or virtual object(s), how the user 118 wore the wearable computing device 104 when looking at the physical object(s) 106 or virtual object(s), which features of the virtual object(s) the user 118 interacted with (e.g., such as whether the user 118 engaged with the virtual object), and any suitable combination thereof. To enhance the interactivity with the physical object(s) 106 and/or virtual objects, the wearable computing device 104 receives a visualization content dataset related to the analytics data. The wearable computing device 104, via the display device 114, then generates a virtual object with additional or visualization features, or a new experience, based on the visualization content dataset.

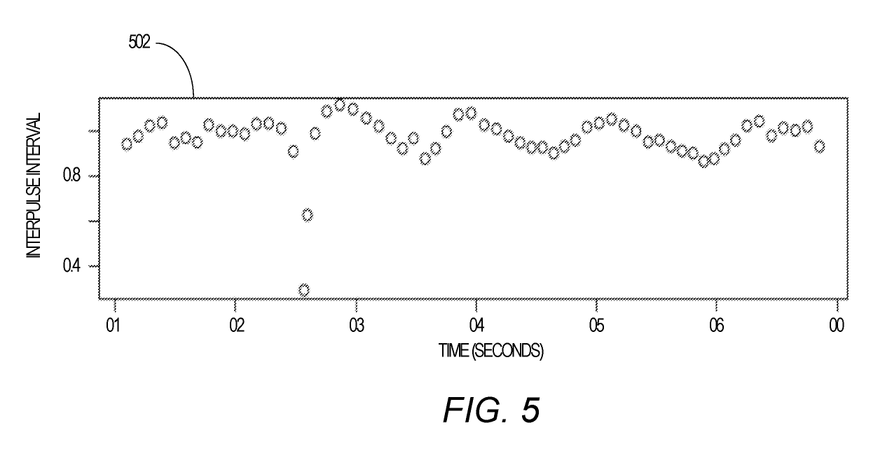

Any of the machines, databases, or devices shown in FIG. 1 may be implemented in a general-purpose computer modified (e.g., configured or programmed) by software to be a special-purpose computer to perform one or more of the functions described herein for that machine, database, or device. For example, a computer system able to implement any one or more of the methodologies described herein is discussed below with respect to FIG. 5. As used herein, a “database” is a data storage resource and may store data structured as a text file, a table, a spreadsheet, a relational database (e.g., an object-relational database), a triple store, a hierarchical data store, or any suitable combination thereof. Moreover, any two or more of the machines, databases, or devices illustrated in FIG. 1 may be combined into a single machine, and the functions described herein for any single machine, database, or device may be subdivided among multiple machines, databases, or devices.

The network 108 may be any network that facilitates communication between or among machines (e.g., server 110), databases, and devices (e.g., device 101). Accordingly, the network 108 may be a wired network, a wireless network (e.g., a mobile or cellular network), or any suitable combination thereof. The network 108 may include one or more portions that constitute a private network, a public network (e.g., the Internet), or any suitable combination thereof.

FIG. 2 is a block diagram of the components of the biometric sensor 116 according to an example embodiment. In one embodiment, the biometric sensor 116 includes one or more processors 202, a photosensor 204, a communication interface 206, and a machine-readable memory 208.

The one or more processors 202 may be any type of commercially available processor, such as processors available from the Intel Corporation, Advanced Micro Devices, Qualcomm, Texas Instruments, or other such processors. Further still, the one or more processors 202 may include one or more special-purpose processors, such as a Field-Programmable Gate Array (FPGA) or an Application Specific Integrated Circuit (ASIC). The one or more processors 202 may also include programmable logic or circuitry that is temporarily configured by software to perform certain operations. Thus, once configured by such software, the one or more processors 202 become specific machines (or specific components of a machine) uniquely tailored to perform the configured functions and are no longer general-purpose processors.

The photosensor 204 is configured to generate a light beam and output a voltage corresponding to the amount of reflected or, in another example embodiment, transmitted, light that is detected by the photosensor 204. In particular, the photosensor 204 emits a light beam into the skin of user 104. As light strikes the user's 104 body tissue, it is absorbed, reflected, and, potentially, transmitted. The amount of blood in the body tissue affects the amount of light reflected or transmitted—the larger the irradiated blood volume, the lower the amount of light reflected or transmitted. As the blood volume in the arteries change with the cardiac cycle (e.g., through expansion and contraction), the user's 104 heart rate can be measured indirectly from the changes in the amount of light reflected or transmitted. This optical measurement of the change of blood volume in the blood vessels is referred to as photoplethysmography (PPG). As discussed above, the photosensor 204 may be in direct contact with the user's 104 skin, such as on the wrist, fingers, or forehead.

In one embodiment, the light emitted from the photosensor 204 has an approximate wavelength of 495-570 nanometers (nm) (e.g., green light). In another embodiment, the light emitted from the photosensor 204 has an approximate wavelength of 620-750 nm (e.g., red light). In alternative embodiments, the wearable computing device 104 includes one or more biometric sensors 116 that include different sources of light such that any given biometric sensor 116 may emit a green or red light depending on the form factor of the wearable computing device 104 or where the wearable computing device 104 is placed on the user's 104 body. One example of a photosensor 204 that may be used by the biometric sensor 116 includes the BioMon Sensor SFH 7050, which is available from OSRAM Opto Semiconductors Inc., located in Sunnyvale, Calif.

The communication interface 206 is configured to facilitate electronic communications between the biometric sensor 116, the wearable computing device 104, and/or the display device 114. The communication interface 206 may include one or more wired communication interfaces (e.g., Universal Serial Bus (USB), an I²C bus, an RS-232 interface, an RS-485 interface, etc.), one or more wireless transceivers, such as a Bluetooth® transceiver, a Near Field Communication (NFC) transceiver, an 802.11x transceiver, a 3G (e.g., a GSM and/or CDMA) transceiver, a 4G (e.g., LTE and/or Mobile WiMAX) transceiver, or combinations of wired and wireless interfaces and transceivers. In one embodiment, the communication interface 206 communicates data 212, such as the determined heartrate 234, to the wearable computing device 104 and/or the display device 114. The biometric sensor 116 may also receive instructions and/or calibration data from the wearable computing device 104 via the communication interface 206. For example, the wearable computing device 104 may provide the biometric sensor 116 with information about the user 104, such as the user's 104 height, weight, age, or other such information.

The machine-readable memory 208 includes various modules 210 and data 212 for implementing the features of the biometric sensor 116. The machine-readable memory 208 includes one or more devices configured to store instructions and data temporarily or permanently and may include, but not be limited to, random-access memory (RAM), read-only memory (ROM), buffer memory, flash memory, optical media, magnetic media, cache memory, other types of storage (e.g., Erasable Programmable Read-Only Memory (EEPROM)) and/or any suitable combination thereof. The term “machine-readable memory” should be taken to include a single medium or multiple media (e.g., a centralized or distributed database, or associated caches and servers) able to store the modules 210 and the data 212. Accordingly, the machine-readable memory 208 may be implemented as a single storage apparatus or device, or, alternatively and/or additionally, as “cloud-based” storage systems or storage networks that include multiple storage apparatus or devices. As shown in FIG. 2, the machine-readable memory 208 excludes signals per se.

In one embodiment, the modules 210 are written in a computer-programming and/or scripting language. Examples of such languages include, but are not limited to, C, C++, C#, Java, JavaScript, Perl, Python, Ruby, or any other computer programming and/or scripting language now known or later developed.

The modules 210 include one or more modules 214-222 that implement the features of the biometric sensor 116. In one embodiment, the modules include a signal filter module 214, a decimation module 216, a normalization module 218, a slope determination module 220, and an interpulse interval module 222. The data 212 includes one or more different sets of data 212 used by, or in support of, the modules 210. In one embodiment, the data 212 includes one or more measured voltages 224, one or more filtered voltages 226, one or more normalized voltages 228, one or more determined slopes 230, one or more interpulse intervals 232, and one or more determined heartrates 234.

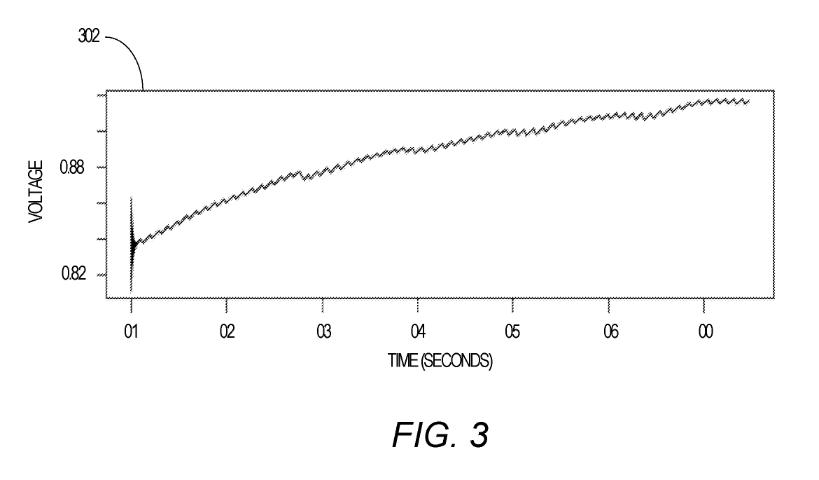

As discussed previously, the photosensor 204 is configured to obtain and/or record one or more voltages corresponding to the measured reflected or transmitted light emitted by the photosensor 204. The voltages obtained by the photosensor 204 may be stored as the measured voltages 224. FIG. 3 illustrates a graph 302 of the measured voltages 224 obtained by the photosensor 204, according to an example embodiment. In one embodiment, the measured voltages 224 are sampled at a predetermined frequency, such as 100 Hz, over a preconfigured time period, such as six seconds. Thus, as shown in FIG. 3, the measured voltages 224 (represented on the Y-axis) are sampled over a period of six seconds (represented on the X-axis). In this embodiment, the photosensor 204 obtains 600 measured voltages 224.

The measured voltages 224 may be divided into one or more sets, depending on the timeframe in which a given voltage was measured. In one embodiment, each measured voltage 224 corresponds to a single timeframe—in other words, the measured voltages 224 include 600 voltages that associated with a first time frame, a second 600 voltages that are associated with a second time frame, which are different from the first 600 voltages, and so forth. In an alternative embodiment, a measured voltage is associated with multiple timeframes according to a rolling window basis. In this alternative embodiment, a given measured voltage at position Vₜ for a timeframe t corresponds to a measured voltage at position Vₜ₋ₙ for the nth timeframe. Thus, in this alternative embodiment, a given measured voltage may be identifiable across multiple timeframes.

In alternative embodiments, the photosensor 204 may be configured to obtain more or fewer such measured voltages. For example, the predetermined frequency (e.g., the predetermined sampling rate) and/or the preconfigured time period may be configurable via the wearable computing device 104 such that either measurements may be increased or decreased according to one or more inputs provided by the user 104. Alternatively, and/or additionally, the wearable computing device 104 may automatically increase or decrease either measurement according to the determined heartrate(s) 234 or the variability of the determined heartrate(s).

Referring back to FIG. 2, a signal filter module 214 is configured to filter the measured voltages 224 to obtain the filtered voltages 226. As the measured voltages 224 may be noisy, such as noise being introduced from the transmission of the voltages to the machine-readable memory 208, the biometric sensor 116 may be configured to remove these noisy elements. Accordingly, the signal filter module 214 may implement one or more filters, such as one or more bandpass and/or bandstop filters. In one embodiment, the signal filter module 214 implements a bandpass Infinite Impulse Response (IIR) filter that allows a specified range of frequencies, which may include 1 Hz to 50 Hz, inclusive. Alternatively and/or additionally, the frequencies assigned to the bandpass IIR filter are based on the sampling rate of the measured voltages. In this regard, the frequencies assigned to the bandpass IIR filter are approximately one-half the sampling rate of the measured voltages. Thus, where the measured voltages are sampled at a rate of 60 Hz, the bandpass IIR filter is configured for frequencies in the range of 1 to 30 Hz, inclusive.

The signal filter module 214 may also implement a bandstop IIR filter that removes a specified frequency from the measured voltages 224, such as 60 Hz.

In yet a further embodiment, the range of frequencies permitted by the bandpass filter and the frequency removed by the bandstop are geolocation dependent, where different frequencies for the bandpass filter and different frequencies for the bandstop filter correspond with different geographic locations. In this regard, the biometric sensor 116 may implement a look-up table that assigns the frequencies to the bandpass and/or bandstop filter according to a determined geolocation (e.g., from one or more GPS coordinates received via the communication interface 206). Thus, a first geographic location (e.g., the United States) may result in a first set of frequencies being assigned to the signal filter module 214 and a second geographic location (e.g., the People's Republic of China) may result in a second set of frequencies being assigned to the signal filter module 214, where the first set of frequencies are different from the second set of frequencies. The filtering of the measured voltages 224 by the signal filter module 214 results in the filtered voltages 226.

One of the technical benefits obtained by implementing the signal filter module 214 as an IIR filter is that the signal filter module 214 can achieve a given filtering characteristic using less memory and calculations than a similar Finite Impulse Response filter. With limited electric power and computing resources, like the biometric sensor 116, having a lightweight and resource sensitive filter is a desirable characteristic. Thus, by implementing the signal filter module 214 as an IIR filter, the biometric sensor 116 uses less resources (e.g., electric power and memory) than comparable biometric sensors.

After the filtered voltages 226 are obtained, the biometric sensor 116 may then decimate the filtered voltages 226, via the decimation module 216, to obtain one or more decimated voltages (not shown). Alternatively, the measured voltages 224 may be decimated by the decimation module 216 prior to the filtering performed by the signal filter module 214.

In one embodiment, the decimation module 216 decimates the obtained voltages (e.g., the filtered voltages 226 and/or the measured voltages 224) depending on the location of the biometric sensor 116. For example, where the biometric sensor 116 obtains measurements from the user's 104 head, the decimation module 216 may decimate the voltages by a factor of two.

Alternatively, where the biometric sensor 116 obtains measurements from the user's 104 wrist, the decimation module 216 may decimate the voltages by a factor of three. Additionally, or alternatively, the decimation module 216 may be instructed to forego decimation, such as where the biometric sensor 116 is unable to acquire a predetermined threshold number of measured voltages within the designated timeframe (e.g., 600 measured voltages in six seconds).

In addition, the decimation module 216 may be configured to decimate the obtained voltages (e.g., the filtered voltages 226 and/or the measured voltages 224) in response to a determination of whether minimum computation requirements have been met for the obtained voltages. For example, the decimation module 216 with a set of minimum processor, memory, and/or storage requirements and, in the event that such requirements are not met, the decimation module 216 may perform the decimation of the obtained voltages.

The filtered voltages 226, regardless of being decimated, may then be subject to a median subtraction according to a median voltage obtained from the set of filtered voltages 226. In one embodiment, the median subtraction may be performed by one or more of the modules 210, such as the decimation module 216 and/or the normalization module 218. The median subtraction accounts for the differences in voltages that may be obtained depending on the location of the user's 104 body that the biometric sensor 116 contacts. For example, the voltages obtained from the forehead of the user's 104 body may be different than the voltages obtained from the wrist or forearm of the user's 104 body. In one embodiment, the median subtraction is performed by determining a median voltage from a set of voltages for a given timeframe, and then subtracting said median voltage from each of the voltages within the given timeframe. Accordingly, in this embodiment, it is possible that the new set of voltages will include negative voltages (e.g., where the median voltage exceeds the measured voltage).

The obtained median voltages may then be normalized by a normalization module 218 implemented by the processor(s) 202. In one embodiment, the normalization module 218 generates a set of normalized voltages 228, where the normalized voltages 228 have values between a predetermined minimum (e.g., zero) and a predetermined maximum (e.g., one). For example, the normalization module 218 may normalize each of the obtained median-subtracted voltages based on the minimum voltage and the maximum voltage over a corresponding median-subtracted timeframe. One equation for normalizing the median voltages is:

$${x}_{T}^{′} = \frac{{x}_{T} - min ( {A}_{T} )}{max ( {A}_{T} ) - min ( {A}_{T} )} ,$$

where
T=a selected timeframe;
x=the value of a median-subtracted voltage for the selected timeframe T; and
A=the set of median-subtracted voltages corresponding to the timeframe T.

The voltages obtained in this manner are then stored as the normalized voltages 228.

Following the output of the normalized voltages 228, the biometric sensor 116 then invokes the slope determination module 220. The slope determination module 220 is configured to determine one or more slope(s) of the normalized voltages 228 over a sliding window having a preconfigured duration. In general, a slope is the gradient of a graph, the change in a y variable over a defined segment of the x variable. Here, the x-axis values are time and the y-axis values are voltage. The slope determination module 220 may determine the slopes of the normalized voltages 228 by linear fit.

In one embodiment, the preconfigured duration for the sliding window is 90 milliseconds (ms). In this embodiment, the preconfigured duration may be established as a default or initial duration prior to biometric sensor 116 being used with a particular user 104. However, after the biometric sensor 116 has been used with a particular user 104, the slope determination module 220 may adjust (e.g., increase and/or decrease) the duration of this sliding window. For example, where the number of slopes for a given sliding window duration is less than a threshold amount (e.g., four slopes), the slope determination module 220 may then increase the sliding window duration by a preconfigured amount (e.g., 2 ms, 5 ms, 10 ms, etc.) until the threshold amount of slopes have been determined for the sliding window duration. Similarly, where the number of slopes for a given sliding window duration is greater than a threshold amount (e.g., 15 slopes), the slope determination module 220 may then decrease the sliding window duration by the same, or another, preconfigured amount (e.g., 2 ms, 5 ms, 10 ms, etc.) until the number of slopes determined within the sliding window is at or below this threshold amount.

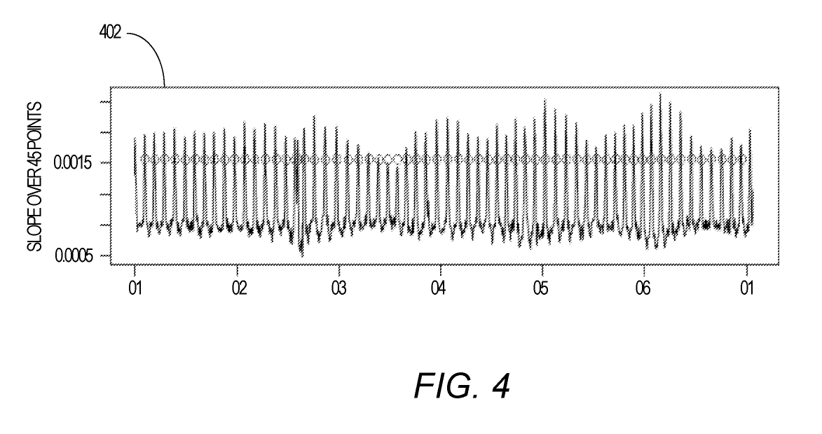

In addition to determining the slopes for the normalized voltages 228, the slope determination module 220 also associates a time index with each of the determined slopes. The determined slopes and their associated time indices are then stored as the determined slopes 230. FIG. 4 illustrates a graph 402 of the determined slopes 230, according to an example embodiment, corresponding to the measured voltages 224 illustrated in the graph 302 of FIG. 3.

Using the determined slopes 230, the biometric sensor 116 then determines a local minima for each of the sliding window sets of determined slopes 230. In one embodiment, the interpulse interval module 222 is configured to determine the local minima for these sliding window sets using a peak detection algorithm on the inverted data. The interpulse interval module 222 then determines the time interval between the slope minima of the sliding window sets using their associated time indices. More particularly, the interpulse interval module 222 determines the time interval between consecutive local minima. These time intervals are then stored as the interpulse intervals 232. FIG. 5 illustrates a graph 502 of the interpulse intervals 232, according to an example embodiment, corresponding to the measured voltages 224 illustrated in the graph 302 and derived from the determined slopes 230 illustrated in the graph 402.

From the interpulse intervals 232, the interpulse interval module 222 determines one or more instantaneous heartrate values for display by the display device 114. In one embodiment, the heartrate values are represented as beats per minute, which the interpulse interval module 222 determines by dividing 60 by each of the interpulse intervals 232. The resulting values from these division operations are then stored as the heartrate(s) 234. Accordingly, the biometric sensor 116 communicates the heartrate(s) 234 to the wearable computing device 104 and/or the display device 114 via the communicate interface 206. The heartrate(s) 234 are then displayed on the display device 114 for viewing by the user 104. In one alternative embodiment, the interpulse interval module 222 further determines a median heartrate from the heartrate(s) 234, and the biometric sensor 116 communicates the median heartrate to the wearable computing device 104 for display by the display device 114.

As one of ordinary skill in the art will understand, the foregoing operations by the various modules 214-222 takes place within seconds of the photosensor 204 acquiring the measured voltages 204. Accordingly, the heartrate(s) 234 determined by the biometric sensor 116 are displayable by the display device 114 within seconds of the photosensor 204 being activated. Thus, unlike conventional techniques for determining a heartrate (e.g., power spectral density analysis), the disclosed biometric sensor 116 can provide the user's 104 heartrate in a much narrower timeframe.

Furthermore, the deployment of multiple biometric sensor(s) 116 can be used to detect more complicated cardiovascular problems, such as arteriosclerosis. For example, a first biometric sensor 116 placed on the user's 104 forehead (e.g., a first wearable computing device 104 is a helmet) and a second biometric sensor 116 placed on the user's 104 wrist (e.g., a second wearable computing device 104 is a watch) can be used to measure pulse wave velocity. As one of ordinary skill in the art will understand, pulse wave velocity is used as a measure of arterial stiffness, which can indicate whether the user 104 has arteriosclerosis. In this embodiment, one or more wearable computing device(s) 104 may be networked and synchronized so as to share the measurements obtained by their respective biometric sensor(s) 116. In another embodiment, the multiple biometric sensor(s) 116 are managed by a single wearable computing device 104.

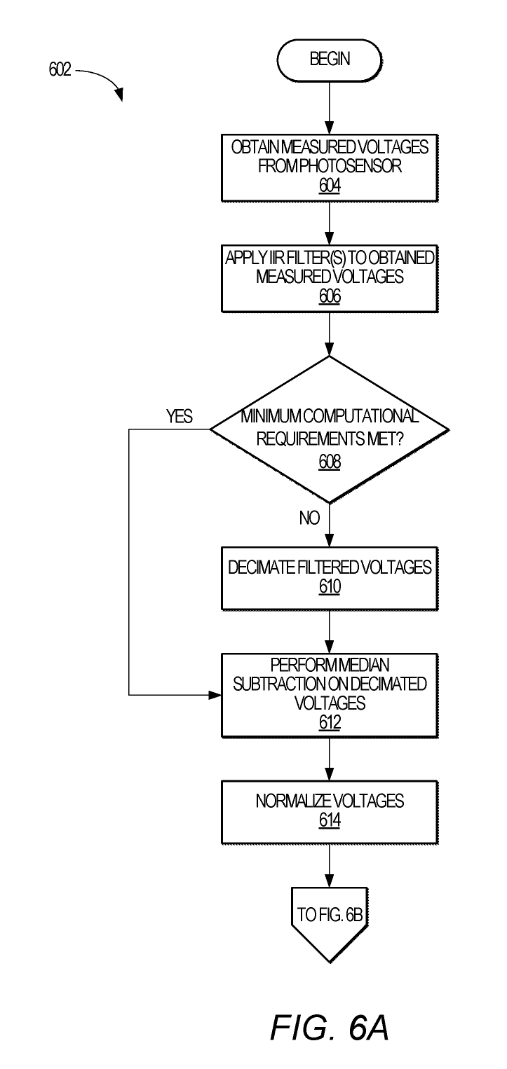

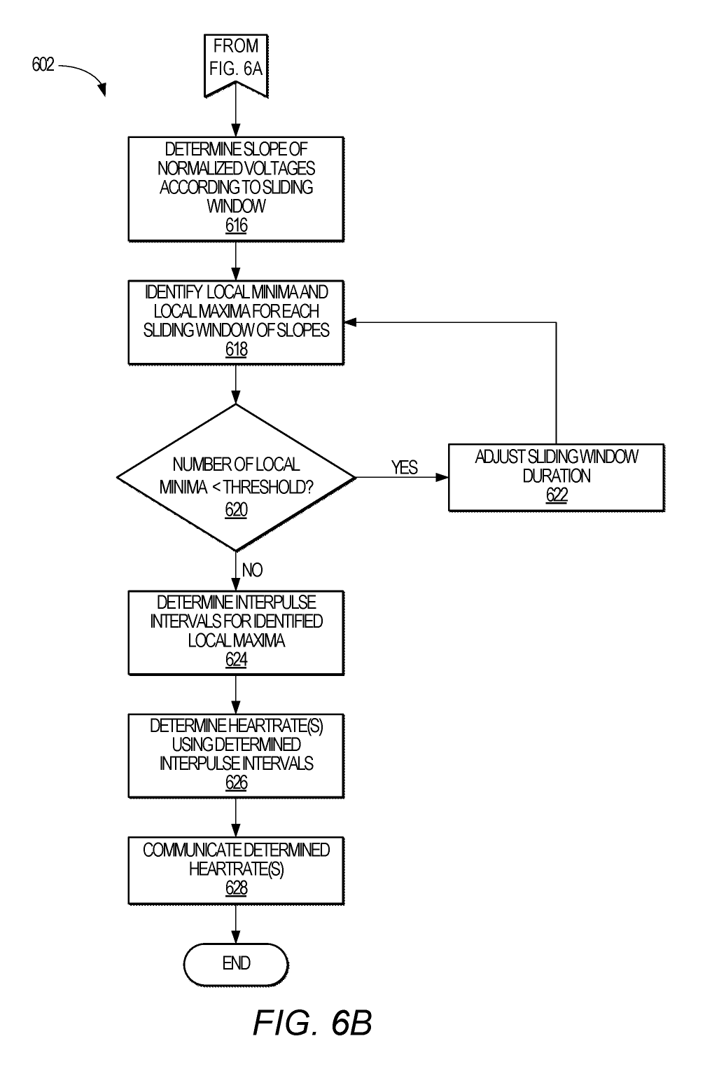

FIGS. 6A-6B illustrate a method 602 for determining a heartrate using the biometric sensor 116 illustrated in FIG. 2, according to an example embodiment. The method 602 may be implemented by one or more components of the wearable computing device 104 and/or the biometric sensor 116 and is discussed by way of reference thereto.

Initially, with reference to FIG. 2 and FIG. 6A, the biometric sensor 116 obtains a plurality of measured voltages 224 (Operation 604). The measured voltages 224 may be obtained by a photosensor 204 of the biometric sensor 116. The biometric sensor 116 then filters the obtained measured voltages 224 to obtain one or more filtered voltages 226 (Operation 606). As also discussed with reference to FIG. 2, the biometric sensor 116 may implement a signal filter module 214 that applies a bandpass and/or bandstop IIR filter to the measured voltages 224 to obtain the filtered voltages 226.

The decimation module 216 then determines whether the biometric sensor 116 meets a minimum set of computing requirements (e.g., processor speed, available volatile memory, available non-volatile memory, etc.) (Operation 608). Where the minimum computing requirements have been met (e.g., the “YES” branch of Operation 608), the method 602 then proceeds to Operation 612. Alternatively, where the minimum computing requirements have not been met (e.g., the “NO” branch of Operation 608), the decimation module 216 then decimates the filtered voltages 226 (Operation 610).

Thereafter, the biometric sensor 116 then performs a median subtraction on the filtered voltages 226, regardless of whether the filtered voltages 226 have been decimated (Operation 612). As explained previously, the median subtraction accounts for the differences in voltages that may be obtained depending on the location of the user's 104 body that the biometric sensor 116 contacts. The median voltages are then normalized via a normalization module 218 (Operation 614) and stored as the normalized voltages 228. In one embodiment, the normalized voltages 228 range between (or including) zero and one.

Referring to FIG. 6B, the biometric sensor 116 then determines slopes of the normalized voltages 228 according to a sliding window having a preconfigured duration (Operation 616). As discussed above with reference to FIG. 2, this duration may be configured at 90 ms. The determined slopes are then stored as the determined slopes 230.

The biometric sensor 116 then identifies one or more local minima for each sliding window of the determined slopes 230 (Operation 618). Furthermore, in one embodiment, the biometric sensor 116 determines whether a sufficient number of identified local minima have been obtained by comparing the number of local minima with a preconfigured threshold (Operation 620). If the number of identified local minima is less than (or equal to) the preconfigured threshold (e.g., “YES” branch of Operation 620), the biometric sensor 116 adjusts the duration of the sliding window by a predetermined amount (Operation 622), such as by increasing the sliding window duration by 2 ms, 5 ms, or other such amount. The method 602 may then return to Operation 618 where the biometric sensor 116 then re-identifies the local minima for the determined slopes 230.

Alternatively, where a sufficient number of identified local minima have been obtained (e.g., “NO” branch of Operation 620), the interpulse interval module 222 then determines the time interval between the local minima (e.g., the time in milliseconds—between consecutive local minima) (Operation 624). The interpulse interval module 222 stores these determined interpulse intervals as the interpulse intervals 232. From the interpulse intervals 232, the interpulse interval module 222 then determines one or more heartrates 234 for a specified time domain (e.g., beats per minute) (Operation 626). The determined one or more heartrates 234 are then communicated to the wearable computing device 104 and/or the display device 114 via the communication interface 206 (Operation 628). The determined heartrates 234 may then be displayed by the display device 114 for viewing by the user 104.

In this manner, the biometric sensor 116 provides a determined heartrate within a timeframe that is significantly faster than conventional methods. Furthermore, the operations performed by the biometric sensor 116 are fast and light-weight, which are well suited for mobile and embedded deployment. In particular, the biometric sensor 116 can be deployed with other CPU- and memory-intensive processes with less impact than alternative sensors with computations in the frequency domain. This is technically beneficial because it means that the biometric sensor 116 can be used in a device, such as the wearable computing device 104, where computing resources (e.g., electric power, CPU cycles, machine-readable memory, etc.) are valued at a premium and are generally needed to perform more intensive computing operations. Furthermore, as the disclosed biometric sensor 116 has a small footprint, both physically and computationally, it can be embedded within the wearable computing device 104 without impacting physical comfort or computational abilities. Thus, the biometric sensor 116 has a number of technical benefits, both physically and computationally.

### Modules, Components, and Logic

Certain embodiments are described herein as including logic or a number of components, modules, or mechanisms. Modules may constitute either software modules (e.g., code embodied on a machine-readable medium) or hardware modules. A “hardware module” is a tangible unit capable of performing certain operations and may be configured or arranged in a certain physical manner. In various example embodiments, one or more computer systems (e.g., a standalone computer system, a client computer system, or a server computer system) or one or more hardware modules of a computer system (e.g., a processor or a group of processors) may be configured by software (e.g., an application or application portion) as a hardware module that operates to perform certain operations as described herein.

In some embodiments, a hardware module may be implemented mechanically, electronically, or any suitable combination thereof. For example, a hardware module may include dedicated circuitry or logic that is permanently configured to perform certain operations. For example, a hardware module may be a special-purpose processor, such as a Field-Programmable Gate Array (FPGA) or an Application Specific Integrated Circuit (ASIC). A hardware module may also include programmable logic or circuitry that is temporarily configured by software to perform certain operations. For example, a hardware module may include software executed by a general-purpose processor or other programmable processor. Once configured by such software, hardware modules become specific machines (or specific components of a machine) uniquely tailored to perform the configured functions and are no longer general-purpose processors. It will be appreciated that the decision to implement a hardware module mechanically, in dedicated and permanently configured circuitry, or in temporarily configured circuitry (e.g., configured by software) may be driven by cost and time considerations.

Accordingly, the phrase “hardware module” should be understood to encompass a tangible entity, be that an entity that is physically constructed, permanently configured (e.g., hardwired), or temporarily configured (e.g., programmed) to operate in a certain manner or to perform certain operations described herein. As used herein, “hardware-implemented module” refers to a hardware module. Considering embodiments in which hardware modules are temporarily configured (e.g., programmed), each of the hardware modules need not be configured or instantiated at any one instance in time. For example, where a hardware module comprises a general-purpose processor configured by software to become a special-purpose processor, the general-purpose processor may be configured as respectively different special-purpose processors (e.g., comprising different hardware modules) at different times. Software accordingly configures a particular processor or processors, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple hardware modules exist contemporaneously, communications may be achieved through signal transmission (e.g., over appropriate circuits and buses) between or among two or more of the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions described herein. As used herein, “processor-implemented module” refers to a hardware module implemented using one or more processors.

Similarly, the methods described herein may be at least partially processor-implemented, with a particular processor or processors being an example of hardware. For example, at least some of the operations of a method may be performed by one or more processors or processor-implemented modules. Moreover, the one or more processors may also operate to support performance of the relevant operations in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces (e.g., an Application Program Interface (API)).

The performance of certain of the operations may be distributed among the processors, not only residing within a single machine, but deployed across a number of machines. In some example embodiments, the processors or processor-implemented modules may be located in a single geographic location (e.g., within a home environment, an office environment, or a server farm). In other example embodiments, the processors or processor-implemented modules may be distributed across a number of geographic locations.

### Example Machine Architecture and Machine-Readable Medium

FIG. 7 is a block diagram illustrating components of a machine 700, according to some example embodiments, able to read instructions from a machine-readable medium (e.g., a machine-readable storage medium) and perform any one or more of the methodologies discussed herein. Specifically, FIG. 7 shows a diagrammatic representation of the machine 700 in the example form of a computer system, within which instructions 716 (e.g., software, a program, an application, an applet, an app, or other executable code) for causing the machine 700 to perform any one or more of the methodologies discussed herein may be executed. For example, the instructions may cause the machine to execute the method illustrated in FIGS. 6A-6B. Additionally, or alternatively, the instructions may implement one or more of the modules 210 illustrated in FIG. 2 and so forth. The instructions transform the general, non-programmed machine into a particular machine programmed to carry out the described and illustrated functions in the manner described. In alternative embodiments, the machine 700 operates as a standalone device or may be coupled (e.g., networked) to other machines. In a networked deployment, the machine 700 may operate in the capacity of a server machine or a client machine in a server-client network environment, or as a peer machine in a peer-to-peer (or distributed) network environment. The machine 700 may comprise, but not be limited to, a server computer, a client computer, a personal computer (PC), a tablet computer, a laptop computer, a netbook, a set-top box (STB), a personal digital assistant (PDA), an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 716, sequentially or otherwise, that specify actions to be taken by machine 700. Further, while only a single machine 700 is illustrated, the term “machine” shall also be taken to include a collection of machines 700 that individually or jointly execute the instructions 716 to perform any one or more of the methodologies discussed herein.

The machine 700 may include processors 710, memory 730, and I/O components 750, which may be configured to communicate with each other such as via a bus 702. In an example embodiment, the processors 710 (e.g., a Central Processing Unit (CPU), a Reduced Instruction Set Computing (RISC) processor, a Complex Instruction Set Computing (CISC) processor, a Graphics Processing Unit (GPU), a Digital Signal Processor (DSP), an Application Specific Integrated Circuit (ASIC), a Radio-Frequency Integrated Circuit (RFIC), another processor, or any suitable combination thereof) may include, for example, processor 712 and processor 714 that may execute instructions 716. The term “processor” is intended to include multi-core processor that may comprise two or more independent processors (sometimes referred to as “cores”) that may execute instructions contemporaneously. Although FIG. 7 shows multiple processors, the machine 700 may include a single processor with a single core, a single processor with multiple cores (e.g., a multi-core process), multiple processors with a single core, multiple processors with multiples cores, or any combination thereof.

The memory/storage 730 may include a memory 732, such as a main memory, or other memory storage, and a storage unit 736, both accessible to the processors 710 such as via the bus 702. The storage unit 736 and memory 732 store the instructions 716 embodying any one or more of the methodologies or functions described herein. The instructions 716 may also reside, completely or partially, within the memory 732, within the storage unit 736, within at least one of the processors 710 (e.g., within the processor's cache memory), or any suitable combination thereof, during execution thereof by the machine 700. Accordingly, the memory 732, the storage unit 736, and the memory of processors 710 are examples of machine-readable media.

As used herein, “machine-readable medium” means a device able to store instructions and data temporarily or permanently and may include, but is not be limited to, random-access memory (RAM), read-only memory (ROM), buffer memory, flash memory, optical media, magnetic media, cache memory, other types of storage (e.g., Erasable Programmable Read-Only Memory (EEPROM)) and/or any suitable combination thereof. The term “machine-readable medium” should be taken to include a single medium or multiple media (e.g., a centralized or distributed database, or associated caches and servers) able to store instructions 716. The term “machine-readable medium” shall also be taken to include any medium, or combination of multiple media, that is capable of storing instructions (e.g., instructions 716) for execution by a machine (e.g., machine 700), such that the instructions, when executed by one or more processors of the machine 700 (e.g., processors 710), cause the machine 700 to perform any one or more of the methodologies described herein. Accordingly, a “machine-readable medium” refers to a single storage apparatus or device, as well as “cloud-based” storage systems or storage networks that include multiple storage apparatus or devices. The term “machine-readable medium” excludes signals per se.

The I/O components 750 may include a wide variety of components to receive input, provide output, produce output, transmit information, exchange information, capture measurements, and so on. The specific I/O components 750 that are included in a particular machine will depend on the type of machine. For example, portable machines such as mobile phones will likely include a touch input device or other such input mechanisms, while a headless server machine will likely not include such a touch input device. It will be appreciated that the I/O components 750 may include many other components that are not shown in FIG. 7. The I/O components 750 are grouped according to functionality merely for simplifying the following discussion and the grouping is in no way limiting. In various example embodiments, the I/O components 750 may include output components 752 and input components 754. The output components 752 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 754 may include alphanumeric input components (e.g., a keyboard, a touch screen configured to receive alphanumeric input, a photo-optical keyboard, or other alphanumeric input components), point based input components (e.g., a mouse, a touchpad, a trackball, a joystick, a motion sensor, or other pointing instrument), tactile input components (e.g., a physical button, a touch screen that provides location and/or force of touches or touch gestures, or other tactile input components), audio input components (e.g., a microphone), and the like.

In further example embodiments, the I/O components 750 may include biometric components 756, motion components 758, environmental components 760, or position components 762 among a wide array of other components. For example, the biometric components 756 may include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram based identification), and the like. The motion components 758 may include acceleration sensor components (e.g., accelerometer), gravitation sensor components, rotation sensor components (e.g., gyroscope), and so forth. The environmental components 760 may include, for example, illumination sensor components (e.g., photometer), temperature sensor components (e.g., one or more thermometer that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 762 may include location sensor components (e.g., a Global Position System (GPS) receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 750 may include communication components 764 operable to couple the machine 700 to a network 780 or devices 770 via coupling 782 and coupling 772 respectively. For example, the communication components 764 may include a network interface component or other suitable device to interface with the network 780. In further examples, communication components 764 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 770 may be another machine or any of a wide variety of peripheral devices (e.g., a peripheral device coupled via a Universal Serial Bus (USB)).

Moreover, the communication components 764 may detect identifiers or include components operable to detect identifiers. For example, the communication components 764 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 764, such as, location via Internet Protocol (IP) geo-location, location via Wi-Fi® signal triangulation, location via detecting a NFC beacon signal that may indicate a particular location, and so forth.

### Transmission Medium

In various example embodiments, one or more portions of the network 780 may be an ad hoc network, an intranet, an extranet, a virtual private network (VPN), a local area network (LAN), a wireless LAN (WLAN), a wide area network (WAN), a wireless WAN (WWAN), a metropolitan area network (MAN), the Internet, a portion of the Internet, a portion of the Public Switched Telephone Network (PSTN), a plain old telephone service (POTS) network, a cellular telephone network, a wireless network, a Wi-Fi® network, another type of network, or a combination of two or more such networks. For example, the network 780 or a portion of the network 780 may include a wireless or cellular network and the coupling 782 may be a Code Division Multiple Access (CDMA) connection, a Global System for Mobile communications (GSM) connection, or other type of cellular or wireless coupling. In this example, the coupling 782 may implement any of a variety of types of data transfer technology, such as Single Carrier Radio Transmission Technology (1×RTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard setting organizations, other long range protocols, or other data transfer technology.

The instructions 716 may be transmitted or received over the network 780 using a transmission medium via a network interface device (e.g., a network interface component included in the communication components 764) and utilizing any one of a number of well-known transfer protocols (e.g., hypertext transfer protocol (HTTP)). Similarly, the instructions 716 may be transmitted or received using a transmission medium via the coupling 772 (e.g., a peer-to-peer coupling) to devices 770. The term “transmission medium” shall be taken to include any intangible medium that is capable of storing, encoding, or carrying instructions 716 for execution by the machine 700, and includes digital or analog communications signals or other intangible medium to facilitate communication of such software.

### Language

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Although an overview of the inventive subject matter has been described with reference to specific example embodiments, various modifications and changes may be made to these embodiments without departing from the broader scope of embodiments of the present disclosure. Such embodiments of the inventive subject matter may be referred to herein, individually or collectively, by the term “invention” merely for convenience and without intending to voluntarily limit the scope of this application to any single disclosure or inventive concept if more than one is, in fact, disclosed.

The embodiments illustrated herein are described in sufficient detail to enable those skilled in the art to practice the teachings disclosed. Other embodiments may be used and derived therefrom, such that structural and logical substitutions and changes may be made without departing from the scope of this disclosure. The Detailed Description, therefore, is not to be taken in a limiting sense, and the scope of various embodiments is defined only by the appended claims, along with the full range of equivalents to which such claims are entitled.

As used herein, the term “or” may be construed in either an inclusive or exclusive sense. Moreover, plural instances may be provided for resources, operations, or structures described herein as a single instance. Additionally, boundaries between various resources, operations, modules, engines, and data stores are somewhat arbitrary, and particular operations are illustrated in a context of specific illustrative configurations. Other allocations of functionality are envisioned and may fall within a scope of various embodiments of the present disclosure. In general, structures and functionality presented as separate resources in the example configurations may be implemented as a combined structure or resource. Similarly, structures and functionality presented as a single resource may be implemented as separate resources. These and other variations, modifications, additions, and improvements fall within a scope of embodiments of the present disclosure as represented by the appended claims. The specification and drawings are, accordingly, to be regarded in an illustrative rather than a restrictive sense.

We claim:

1. A biometric sensor for measuring a heart rate through photoplethysmography, the biometric sensor comprising:
a machine-readable memory storing computer-executable instructions; and
at least one hardware processor in communication with the machine-readable memory that, when the computer-executable instructions are executed, configures the biometric sensor to:
obtain a plurality of voltages in response to a photosensor emitting light into a surface of a human body;
filter at least one predetermined frequency from the plurality of voltages to obtain a plurality of filtered voltages;
normalize the plurality of filtered voltages to obtain a plurality of normalized voltages;
determine a plurality of slopes based on the plurality of normalized voltages;
determine a plurality of local minima based on the determined plurality of slopes;
determine a plurality of interpulse intervals based on the plurality of local maxima, wherein at least one interpulse interval represents a time between a first local minima selected from the plurality of local minima and a consecutive, second local minima selected from the plurality of local minima;
determine at least one heartrate from the determined plurality of interpulse intervals; and
communicate the determined at least one heartrate to a display.

2. The biometric sensor of claim 1, wherein the filter applied to the plurality of voltages comprises a bandpass infinite impulse response filter.

3. The biometric sensor of claim 2, wherein the at least one predetermined frequency of the bandpass infinite impulse response filter comprises a range of frequencies from approximately 1 Hz to approximately 50 Hz.

4. The biometric sensor of claim 1, wherein the biometric sensor is further configured to:
determine a median voltage from the plurality of filtered voltages; and
adjust each voltage of the plurality of filtered voltages by the determined median voltage.

5. The biometric sensor of claim 1, wherein:
the plurality of slopes occur within a preconfigured time duration; and
the preconfigured time duration is changed by a predetermined amount in response to a determination that the number of the plurality of slope minima occurring within the preconfigured time duration is less than a minimum threshold limit or greater than a maximum threshold limit.

6. The biometric sensor of claim 1, wherein the plurality of filtered voltages are decimated by a preconfigured amount.

7. The biometric sensor of claim 1, wherein the plurality of voltages are obtained by the photosensor at a sampling rate of approximately 100 Hz.

8. A method for measuring a heart rate through photoplethysmography, the method comprising:
obtaining, by a photosensor, a plurality of voltages in response to emitting light into a surface of a human body;
filtering, by at least one hardware processor, at least one predetermined frequency from the plurality of voltages to obtain a plurality of filtered voltages;
normalizing, by at least one hardware processor, the plurality of filtered voltages to obtain a plurality of normalized voltages;
determining, by at least one hardware processor, a plurality of slopes based on the plurality of normalized voltages;
determining, by at least one hardware processor, a plurality of local minima based on the determined plurality of slopes;
determining, by at least one hardware processor, a plurality of interpulse intervals based on the plurality of local minima, wherein at least one interpulse interval represents a time between a first local minima selected from the plurality of local minima and a consecutive, second local minima selected from the plurality of local minima;
determining, by at least one hardware processor, at least one heartrate from the determined plurality of interpulse intervals; and
communicating, using at least one communication interface, the determined at least one heartrate to a display.

9. The method of claim 8, wherein the at least one predetermined frequency is filtered from the plurality of voltages by at least one bandpass infinite impulse response filter.

10. The method of claim 9, wherein the at least one predetermined frequency comprises a range of frequencies from approximately 1 Hz to approximate 50 Hz.

11. The method of claim 8, further comprising:
determining a median voltage from the plurality of filtered voltages; and
adjusting each voltage of the plurality of filtered voltages by the determined median voltage.

12. The method of claim 8, wherein:
the plurality of slopes occur within a preconfigured time duration; and
the preconfigured time duration is changed by a predetermined amount in response to a determination that the number of the plurality of slopes occurring within the preconfigured time duration is less than a minimum threshold amount or greater than a maximum threshold amount.

13. The method of claim 8, wherein the plurality of filtered voltages are decimated by a preconfigured amount.

14. The method of claim 8, wherein the plurality of voltages are obtained by the photosensor at a sampling rate of approximately 100 Hz.

15. A machine-readable medium having computer-executable instructions stored thereon that, when executed by at least one hardware processor, causes a biometric sensor to perform a plurality of operations, the plurality of operations comprising:
obtaining a plurality of voltages in response to emitting light into a surface of a human body;
filtering at least one predetermined frequency from the plurality of voltages to obtain a plurality of filtered voltages;
normalizing the plurality of filtered voltages to obtain a plurality of normalized voltages;
determining a plurality of slopes based on the plurality of normalized voltages;
determining a plurality of local minima based on the determined plurality of slopes;
determining a plurality of interpulse intervals based on the plurality of local minima, wherein at least one interpulse interval represents a time between a first local minima selected from the plurality of local minima and a consecutive, second local minima selected from the plurality of local minima;
determining at least one heartrate from the determined plurality of interpulse intervals; and
communicating the determined at least one heartrate to a display.

16. The machine-readable medium of claim 15, wherein the at least one predetermined frequency is filtered from the plurality of voltages by at least one bandpass infinite impulse response filter.

17. The machine-readable medium of claim 16, wherein the predetermined frequency comprises a range of frequencies from approximately 1 Hz to approximate 50 Hz.

18. The machine-readable medium of claim 15, wherein the plurality of operations further comprise:
determining a median voltage from the plurality of filtered voltages; and
adjusting each voltage of the plurality of filtered voltages by the determined median voltage.

19. The machine-readable medium of claim 15, wherein:
the plurality of slopes occur within a preconfigured time duration; and
the preconfigured time duration is increased by a predetermined amount in response to a determination that the number of the plurality of slopes occurring within preconfigured time duration is less than a threshold amount.

20. The machine-readable medium of claim 15, wherein the plurality of voltages are obtained by the photosensor at a sampling rate of approximately 100 Hz.
